(19) United States
(12) Patent Application Publication
(10) Pub. No.: US 2007/0020241 A1
(43) Pub. Date: Jan. 25, 2007

(54) Use of photosensitisation

(76) Inventors: Michael Wilson, London (GB); Sean Nair, London (GB)

Correspondence Address: NIXON & VANDERHYE, PC, 901 NORTH GLEBE ROAD, 11TH FLOOR, ARLINGTON, VA, 22203, US

(21) Appl. No.: 10/575,112

(22) PCT Filed: Oct. 8, 2004
(86) PCT No.: PCT/GB04/04305

§ 371 Date: Jul. 11, 2006

(30) Foreign Application Priority Data
Oct. 9, 2003 (GB) 0322699.9

(51) Int. Cl. A61K 35/76; A61K 31/555; A61K 31/409; A61K 31/5415; A61K 31/538; A61K 31/535; A61K 31/519

(52) U.S. Cl. 424/93.6; 514/185; 514/410; 424/125; 514/250; 514/283; 514/443; 514/34; 514/414; 514/224.8; 514/454; 514/229.8; 514/297; 977/906

(57) ABSTRACT

A composition comprising a conjugate of a photosensitiser and a bacteriophage is provided. The conjugate may be used to kill bacteria, particularly MRSA, EMRSA, VRSA, hetero-VRSA or CA-MRSA in a targeted method of photodynamic therapy.

The present invention relates to a composition comprising a conjugate of a photosensitiser and a bacteriophage, particularly a staphylococcal bacteriophage, known as a staphylophage. The invention also relates to the use of the conjugate in a method of photodynamic therapy for infectious diseases.

### BACKGROUND

The use of antimicrobial agents to counter bacterial infections is becoming increasingly ineffective, due to the rapid emergence of antibiotic resistance amongst many species of pathogenic bacteria. One such pathogen is Staphylococcus aureus (S. aureus), which characteristically causes skin infections such as boils, carbuncles and impetigo, as well as infecting acne, burns and wounds. If the infecting organism is a toxic strain, such infections, or colonised tampons, may give rise to a life-threatening toxaemia known as toxic shock syndrome. The organism may also gain access to the bloodstream from these infections, or from foreign bodies such as intravenous catheters, and so cause infections at other sites, such as endocarditis, osteomyelitis, meningitis and pneumonia

A number of bacteria are responsible for infection of skin and wounds, for example, coagulase-negative staphylococci, Staphylococcus aureus, streptococci, Corynebacterium spp., E. coli, Klebsiella aerogenes, Klebsiella pneumoniae, Enterobacter aerogenes, Propionibacterium acnes, Bacteroides spp., Pseudomonas aeruginosa and Peptostreptococcus spp. Increasingly, these bacteria are showing resistance to antibiotic treatment.

In particular, resistant strains of S. aureus have emerged. Methicillin-resistant S. aureus (MRSA) was first reported in 1961 (Jevons, M. (1961) British Medical Journal, 1, 124-5), and these strains are now a major cause of hospital-acquired infection throughout the world, as well as being prevalent in many nursing and residential homes. This poses an alarming challenge to healthcare, causing significant infection and morbidity of hundreds of patients in the UK each year (Ayliffe et al, J Hosp Infect (1988), 39, 253-90).

Since the first report of MRSA, these organisms have demonstrated resistance to a wide variety of antimicrobials including erythromycin, aminoglycosides, tetracyclines, trimethoprim, sulphonamides and chloramphenicol. MRSA strains have developed that are only susceptible to a single class of clinically-available antibiotics: the glycopeptides such as vancomycin and teicoplanin. However, resistance is developing even to these, as strains tolerant to vancomycin have now been reported (Hiramatsu, K. (1998) American Journal of Medicine, 104, 7S-10S). These strains are variously known as VRSA (Vancomycin resistant Staphylococcus aureus) and hetero-VRSA (resistant strains arising from exposure to high levels of vancomycin). At present, the management of patients with MRSA infections usually involves the administration of antimicrobial agents and again, there is evidence of the development of resistance to many of the agents used.

Due to the emergence of strains which are resistant to virtually all currently-available antimicrobials, MRSA is now a serious threat to health. The term MRSA itself now more accurately applies to methicillin and multiple antimicrobial-resistant S. aureus.

Certain strains of MRSA have been found to spread rapidly not only within hospitals, but also between them. These strains have been termed epidemic MRSA (EMRSA). Since the first EMRSA strain (EMRSA-1) was reported in 1981, 17 distinct EMRSA strains have been identified, all of which are resistant to a number of antimicrobials. Recently, the two most prevalent strains have been EMRSA-15 and -16, which account for 60-70% of the 30000 MRSA isolates reported (Livermore, D (2000) Int. J. Antimicrobial Agents, 16, S3-S10). Importantly, strains of MRSA, (known as community-acquired MRSA (CA-MRSA)) have also started to spread in the community, ie. amongst non-hospitalised individuals.

It is clear from the above that alternative methods of countering bacterial infection, particularly infection with MRSA, are urgently required.

One approach has been to employ a light-activated agent to achieve lethal photosensitization of the organism. This involves treating the organism with a light-activatable chemical (photosensitiser) which, upon irradiation with light of a suitable wavelength, generates cytotoxic species, resulting in bacteriolysis. This technique has been used to achieve killing of a wide range of bacteria, including S. aureus and MRSA strains, in vitro using toluidine blue O (TBO) and aluminium disulphonated phthalocyanine (AlPcS₂) as photosensitisers. Neither photosensitiser nor laser light alone exerted a bacteriocidal effect (Wilson et al, (1994) J Antimicrob Chemother 33, 619-24). In a subsequent study, 16 strains of EMRSA were found to be susceptible to killing by low doses of red light (674 mm) in the presence of AlPcS₂ (Griffiths et al, (1997) J Antirnicrob Chemother, 40, 873-6). At higher light doses, 100% killing was achieved.

Photodynamic therapy (PDT) is the application of such an approach to the treatment of disease. It is an established procedure in the treatment of carcinoma and forms the basis of a means of sterilising blood products. It has only been more recently that the application of PDT to the treatment of infectious diseases has been evaluated. For example, haematoporphyrins in conjunction with an argon laser have been used to treat post-neurosurgical infections and brain abscesses (Lombard et al, (1985), Photodynamic Therapy of Tumours and other Diseases, Ed. Jori & Perria).

One potential problem associated with PDT of infectious diseases is its lack of specificity. Hence, if the photosensitiser binds to, or is taken up by, a host cell, as well as the target organism, then subsequent irradiation may also lead to the death of the host cell. A way to overcome this is by the use of targeting compounds: that is, any compound that is capable of specifically binding to the surface of the pathogen.

Several targeting compounds have previously been shown to be successful in eliminating specific strains of bacteria when they were conjugated to a photosensitiser. For example, immunoglobulin G (IgG) has been used to target S. aureus Protein A (Gross et al (1997), Photochemistry and Photobiology, 66, 872-8), monoclonal antibody against Porphyromonas gingivalis lipopolysaccharide (Bhatti et al (2000), Antimicrobial Agents and Chemotherapy, 44, 2615-8) and poly-L-lysine peptides against P. gingivalis and Actinomyces viscosus (Soukos et al (1998), Antimicrobial Agents and Chemotherapy, 42, 2595-2601). A monoclonal antibody conjugated via dextran chains to the photosensitiser tin (IV) chlorin e6 (SnCe6) was selective for killing P. aeruginosa when exposed to light at 630 nm, leaving S. aureus unaffected (Friedberg et al (1991), Ann N Y Acad Sci, 618, 383-393).

The present inventors have used IgG conjugated to SnCe6 to target EMRSA strains 1, 3, 15 and 16 (Embleton et al (2002), J Antimicrob Chemother, 50, 857-864), achieving higher levels of killing than the photosensitiser alone, and selectively killing the EMRSA strains in a mixture with Streptococcus sanguis. However, a limitation of IgG is that only strains of S. aureus expressing Protein A can be targeted. Hence alternative targeting agents that can target any S. aureus strain are desirable.

Bacteriophage are viruses that infect certain bacteria, often causing them to lyse and hence effecting cell death. They have been proposed as antibacterial agents in their own right. However, one of the problems with using staphylococcal bacteriophage (termed staphylophage) in the treatment of S. aureus disease is their restricted host range. Although there are polyvalent staphylophage which can lyse many S. aureus strains, other strains are resistant and hence bacteriophages alone could not provide an effective method of killing all strains of S. aureus.

It is known that although some bacteriophage will only kill a limited range of bacteria, they will bind to a broader range of bacteria. The present inventors have now found that some bacteriophage can serve as an effective, targeted delivery system for photosensitisers.

The present inventors have found that when a bacteriophage is linked to a photosensitiser, the photosensitiser-bacteriophage conjugate formed is highly effective in killing bacteria when irradiated with light of a suitable wavelength.

Bacteriophage-photosensitiser conjugates could be used to treat or prevent a broad range of bacterial skin and wound infections. The most frequently isolated organisms from skin and wound infections are: coagulase-negative staphylococci, S. aureus, streptococci, e.g. Streptoccocus pyogenes, Corynebacterium spp., E coli, Klebsiella aerogenes, Klebsiella pneumoniae, Enterobacter aerogenes, Propionibacterium acnes, Bacteroides spp., Pseudomonas aeruginosa and Peptostreptococcus spp.

In particular, conjugates of photosensitiser and staphylophage can be used in a method of photodynamic therapy against strains of staphylococci spp, particularly against MRSA, EMRSA, VRSA, hetero-VRSA and CA-MRSA.

The invention provides a composition comprising a photosensitizing compound (photosensitiser) linked to a bacteriophage to form a photosensitiser-bacteriophage conjugate. The bacteriophage may be a staphylococcal phage, and is preferably a staphylophage that can bind to Staphylococcus aureus, particularly MRSA, EMRSA, VRSA, hetero-VRSA or CA-MRSA. The composition may be used in a method of photodynamic therapy.

The bacteriophage is preferably linked to the photosensitiser using a covalent linkage. The photosensitiser and/or the bacteriophage contain or may be modified to contain groups which can be covalently crosslinked using chemical or photoreactive reagents, to produce crosslinked bonds, for example thiol-thiol crosslinking, amine-amine crosslinking, amine-thiol crosslinking, amine-carboxylic acid crosslinking, thiol-carboxylic acid crosslinking, hydroxyl-carboxylic acid crosslinking, hydroxyl-thiol crosslinking and combinations thereof.

The photosensitiser is suitably chosen from porphyrins (e.g. haematoporphyrin derivatives, deuteroporphyrin), phthalocyanines (e.g. zinc, silicon and aluminium phthalocyanines), chlorins (e.g. tin chlorin e6, poly-lysine derivatives of tin chlorin e6, m-tetrahydroxyphenyl chlorin, benzoporphyrin derivatives, tin etiopurpurin), bacteriochlorins, phenothiaziniums (e.g. toluidine blue, methylene blue, dimethylmethylene blue), phenazines (e.g. neutral red), acridines (e.g. acriflavine, proflavin, acridine orange, aminacrine), texaphyrins, cyanines (e.g. merocyanine 540), anthracyclins (e.g. adriamycin and epirubicin), pheophorbides, sapphyrins, fullerene, halogenated xanthenes (e.g. rose bengal), perylenequinonoid pigments (e.g. hypericin, hypocrellin), gilvocarcins, terthiophenes, benzophenanthridines, psoralens and riboflavin.

The invention is directed to killing bacteria using the above-described conjugates. The bacteriophage used in the conjugate may be selected according to the particular organism to be killed, in order to arrive at the conjugate most effective against the particular infecting bacteria. In a preferred embodiment, the infecting bacterium is MRSA, EMRSA, VRSA, hetero-VRSA or CA-MRSA and the conjugate includes the staphylococcal phage 75 or phage φ11.

Table 1 below shows some examples of bacteria-bacteriophage pairs, although many more examples exist. Further novel bacteriophages can be isolated and/or adapted to the target bacteria. The specificity of the treatment can be modified as required by using monovalent bacteriophages, polyvalent bacteriophages or combinations of monovalent bacteriophages or combinations of monovalent and polyvalent bacteriophages.

TABLE 1

| Bacterium | Bacteriophage |
|---|---|
| Staphylococcus aureus | 53, 75, 79, 80, 83, φ11, φ12, φ13, |
| | φ147, φMR11 |
| Staphylococcus epidermidis | 48, 71, numerous (182 different |
| | phage) |
| Staphylococcus spp | φ812, SK311, φ131, SB-I and U16 |
| Streptococcus spp | C₁, SF370.1, SP24, SFL, A1 |
| | (ATCC 12202-B1) various |
| Corynebacterium spp | φ304L φ304S, φ15, φ16, 782 |
| Klebsiella aerogenes and | P1clr100KM |
| Klebsiella pneumoniae | |
| E coli | P1, T1, T3, T4, T7 MS2 |
| Enterobacter aerogenes | Various, P1, M13 |
| Pseudomonas aeruginosa | UNL-1, ACQ, UT1, tba1D3, E79, |
| | F8 & pf20 B3, F116, G101, B86; |
| | T7M, ACq, UT1, BLB, PP7 |
| Propionibacterium acnes | Various, including ATCC |
| | 29399-B1 |
| Bacteroides spp | B40-8 |
| Numerous Gram-negative bacteria P1 | Various |

The composition of the invention suitably comprises at least 0.01 μg/ml, of the photosensitiser, preferably at least 0.02 μg/ml, more preferably at least 0.05 μg/ml up to 200 μg/ml, preferably up to 100 μg/ml, more preferably up to 50 μg/ml. The amount of the bacteriophage in the composition is suitably from 1×10⁵ to 1×10¹⁰ pfu, preferably from 1×10⁶ to 1×10⁹ pfu, more preferably from 1×10⁶ to 1×10 ⁸ pfu.

The composition of the invention may further comprise a source of divalent ions, e.g. Ca²⁺ or Mg²⁺, preferably Ca²⁺. Examples include calcium chloride, calcium carbonate and magnesium chloride. The ions are suitably present in an amount of from 5 to 200 mM, preferably from 5 to 15 mM, more preferably about 10 mM.

The composition may further comprise one or more ingredients chosen from buffers, salts for adjusting the tonicity, antioxidants, preservatives, gelling agents and remineralisation agents.

The invention further provides a method of killing bacteria, comprising

(a) contacting an area to be treated with the composition of the invention such that any bacteria in the area bind to the photosensitiser-bacteriophage conjugate; and

(b) irradiating the area with light at a wavelength absorbed by the photosensitiser.

Suitably the bacteria are as set out above in Table 1, preferably Staphylococcus aureus, more preferably MRSA, EMRSA, VRSA, hetero-VRSA or CA-MRSA.

In the method of the invention, any light source that emits light of an appropriate wavelength may be used. The wavelength of the light is selected to correspond to the absorption maximum of the photosensitiser and to have sufficient energy to activate the photosensitiser. The source of light may be any device or biological system able to generate monochromatic or polychromatic light. Examples include laser, light emitting diode, arc lamp, halogen lamp, incandescent lamp or an emitter of bioluminescence or chemiluminescence. In certain circumstances, sunlight may be suitable. Preferably, the wavelength of the light emitted by the light source may be from 200 to 1060 nm, preferably from 400 to 750 nm. A suitable laser may have a power of from 1 to 100 mW and a beam diameter of from 1 to 10 mm. The light dose for laser irradiation is suitably from 5 to 333 J cm⁻², preferably from 5 to 30 J cm⁻² for laser light. For white light irradiation, a suitable dose is from 0.01 to 100 kJ/cm², preferably from 0.1 to 20 kJc/m², more preferably from 3 to 10 kJ/Cm². The duration of irradiation is suitably from one second to 15 minutes, preferably from 1 to 5 minutes.

The following light sources may be suitable for use in the present invention:
Helium neon (HeNe) gas laser (633 nm)
Argon-pumped dye laser (500-700 nm, 5W output)
Copper vapour-pumped dye laser (600-800 nm)
Excimer-pumped dye laser (400-700 nm)
Gold vapour laser (628 nm, 10 W output)
Tunable solid state laser (532-1060 nm), including Sd:YAG
Light emitting diode (LED) (400-800 nm)
Diode laser (630-850 nm, 25 W output), eg. gallium selenium arsenide
Tungsten filament lamp
Halogen cold light source
Fluorescent lamp.

In the method of the invention, the composition is suitably in the form of a solution or a suspension in a pharmaceutically acceptable aqueous carrier, but may be in the form of a solid such as a powder or a gel, an ointment or a cream. The composition may be applied to the infected area by painting, spreading, spraying or any other conventional technique.

The invention further provides the use of the composition for treatment of the human or animal body. Suitably, the composition is provided for use in the treatment of conditions resulting from bacterial infection, particularly by staphylococci, more particularly by MRSA, EMRSA, VRSA, hetero-VRSA or CA-MRSA.

The invention may be used to treat bacterial infection, particularly by staphylococcal bacteria, more particularly by MRSA, EMRSA, VRSA, hetero-VRSA or CA-MRSA to treat or prevent skin infections such as boils, carbuncles, mastitis and impetigo, to treat or prevent infections of acne, burns or wounds, or to treat or prevent endocarditis, osteomyelitis, meningitis and pneumonia, arising as a result of bacterial infection, to treat or prevent infections arising from the use of catheters, implants or other medical devices, or to prevent infection following an operation, such as a Caesarean section.

The invention may also be used in the prevention of carriage of the bacteria by carriers who themselves show few, if any, symptoms.

### DESCRIPTION OF THE FIGURES

FIG. 1 shows the effect of a phage 75-SnCe6 conjugate on different EMRSA strains.

FIG. 2 shows the effects of conjugate, no conjugate, photosensitiser only or phage only and presence or absence of irradiation on EMRSA-16 and S. epidermidis.

FIGS. 3 to 5 show the effect of the invention on EMRSA-16 and S. aureus 8325-4, varying the light dose.

FIG. 6 shows the effect of light dose using a fixed concentration of Φ11-SnCe6 conjugate on EMRSA-16.

FIG. 7 shows the effect of the invention on strains of VRSA (Mu3), hetero-VRSA (Mu50) and CA-MRSA (MW2).

FIG. 8 shows the effect of the invention on Streptococcus pyogenes.

FIG. 9 shows the effect of the invention on Propionibacterium acnes.

### EXAMPLES

Materials and Methods

The following media were prepared:

Nutrient Broth 2 (NB2) Medium

One litre of medium was made by adding 25 g of Nutrient Broth 2 (Oxoid) (10.0 g/l Lab-Lemco powder, 10.0 g/l peptone, 5.0 g/l NaCl) to 1 litre of deionised, distilled water. After mixing, the medium was autoclaved at 121° C. for 15 min.

Tryptone Soya Yeast Broth (TSY)

One litre of medium was made by adding 39 g of Tryptone Soya Broth (Oxoid) (17.0 g/l pancreatic digest of casein, 3.0 g/l papaic digest of soybean meal, 2.5 g/l glucose, 2.5 g/l di-basic potassium phosphate, 5.0 g/l NaCl) and 0.5% of yeast extract (9.8 g/l total nitrogen, 5.1 g/l amino nitrogen, 0.3 g/l NaCl) to 1 litre of deionised, distilled water. After mixing, the medium was autoclaved at 121° C. for 15 min.

Nutrient Broth 2 Top Agar

0.35% (w/v) of Agar Bacteriological (Agar No. 1, Oxoid) was added to NB2 medium. After mixing, the medium was autoclaved at 121° C. for 15 min.

Nutrient Broth 2 Bottom Agar

0.7% (w/v) of Agar Bacteriological was added to NB2 medium. After autoclaving, 10 mM of CaCl₂ was added (10 ml 1M CaCl₂ in 1 litre of NB2).

Columbia Blood Agar (CBA)

37.1 g of Columbia Agar Base (Oxoid) (23.0 g/l special peptone, 1.0 g/l starch, 5.0 g/l NaCl, 10.0 g/l agar) was added to 1 litre of deionised, distilled water. After autoclaving, the liquid agar was allowed to cool at room temperature until cool enough to handle. 5% (v/v) defibrinated horse blood (E & O Laboratories, Scotland) was then added.

Mannitol Salt Agar (MSA)

111 g of Mannitol Salt Agar (Oxoid) (75.0 g/l NaCl, 10.0 g/l mannitol, 1.0 g/l Lab-lemco powder, 10.0 g/l peptone, 0.025 g/l phenol red, 15.0 g/l agar) was added to 1 litre of deionised, distilled water.

All mixtures were autoclaved at 121° C. for 15 min. The liquid agar was then poured into plates, covered and allowed to cool overnight.

Target Organisms

The organisms used in the examples were as follows, given as names and NCTC (National Collection of Type Cultures, UK) or ATCC (American Type Culture Collection, USA) numbers:
Epidemic methicillin-resistant S. aureus (EMRSA)-1 (NCTC 11939)
EMRSA-3 (NCTC 13130)
EMRSA-15 (NCTC 13142)
EMRSA-16 (NCTC 13143)
Mu3 (ATCC 700698), is a methicillin-resistant Staphylococcus aureus (MRSA) strain with heterogeneous resistance to vancomycin, designated heterogeneously vancomycin-resistant Staphylococcus aureus (hetero-VRSA) (Hanaki et al (1998). J. Anitimicrob. Chemother. 42:199-209)
Mu50 is the archetypal VRSA strain (Hiramatsu et al (1997). J. Antimicrob. Chemother. 40:135-136)
MW2 is a Community-acquired MRSA strain. Community acquired MRSA strains (CA-MRSA) share the presence of staphylococcal cassette chromosome mec (SCCmec) type IV in their genomes, are frequently virulent, and predominantly cause skin and soft tissue infections. The genome sequence of the prototypic CA-MRSA strain, MW2, has revealed the presence of additional virulence factors not commonly present in other S. aureus strains (Baba et al (2002), Lancet. 25;359(9320):1819-27).
Staphylococcus epidermidis (NCTC 11047)
Streptococcus pyogenes (ATCC 12202)
Propionibacterium acnes (ATCC 29399)
Staphyloccus aureus 8324-5 (Novick (1967) Virology 33; 156-166).

All were maintained by weekly subculture on CBA.

Bacteriophage

Phage 75 (Public Health Laboratory Service, UK) is a serogroup F staphylococcal phage, capable of infecting EMRSA-16, EMRSA-3 and weakly infecting EMRSA-15.

Bacteriophage φ11 (Iandolo et al, (2002), Gene 289 (1-2); 109-118) is a temperate bacteriophage of serological group B. φ11 is a transducing phage with a low lysogenisation frequency. It infects S.aureus lytic group III strains which include many human and animal pathogens.

Bacteriophage Propagation

Mid-exponential EMRSA-16 (300 μl) was added to 15 ml Falcon tubes. Approximately 10⁵ pfu of phage 75 were added to the tubes and allowed to incubate at room temperature for 30 min to allow the phage to infect the bacteria. 9 ml of cooled molten top NB2 agar (with 10 mM CaCl₂), was added to the tubes, and the mixture poured onto undried NB2 base agar plates. The plates were left to incubate at 37° C. overnight.

The next morning 1 ml of NB2 with 10 mM CaCl₂ was added to each plate, and the top agar with the liquid medium was scraped into a small centrifuge tube. The collected agar was then spun in a centrifuge at 15000 rpm for 15 min at 4° C. The supernatant was collected and passed through a 0.45 μm (Nalgene) filter to remove any bacterial cells. The resulting solution of phage 75 was stored at 4° C.

Bacteriophage Precipitation

Phage precipitation was carried out to purify the phage 75 from the NB2 medium after propagation. To 5 ml of phage 75 in NB2, 1.3 ml of 5M NaCl (1M final concentration) and 0.2 ml 1× phosphate buffered saline (PBS) (8.0 g/l NaCl, 0.2 g/l KCl, 1.15 g/l Na₂HPO₄, 0.2 g/l KH₂PO₄) were added, and 20% PEG (polyethylene glycol 8000, Sigma) was added to the solution and stirred slowly overnight until completely dissolved. The solution was then placed on ice overnight and the next morning the solution was centrifuged at 8000 rpm for 20 min at 4° C. The supernatant was removed and the remaining pellet was resuspended in 2.5 ml 1× PBS, and filtered through a 0.45 μm filter.

Photosensitiser

The photosensitiser used was tin (IV) chlorin e6 (SnCe6) (Frontier Scientific, Lancashire, UK), which is photoactivatable at 633 nm.

Preparation of Conjugate

2 mg of SnCe6 was dissolved with stirring in 800 μl of activation buffer (0.1 M MES (2-(N-morpholino(ethanesulphonic acid) (Sigma)), 0.5 M NaCl, pH 5.5). An EDC (1-ethyl-3-(3-dimethylaminopropyl)carbodiimide hydrochloride) (Sigma) solution (4 mg in 1 ml activation buffer) and a S—NHS (N-hydroxysulphosuccinimide) (Fluka) solution (2.7 mg in 250 μl activation buffer) were made.

To the dissolved SnCe6, 200 μl of dissolved EDC and S—NHS were added, and the mixture was left for 1 to 4 hours at room temperature with stirring to provide a stable amine-reactive intermediate. The mixture was covered in aluminium foil as SnCe6 is a light sensitive reagent. The reaction was quenched by adding 1.4 μl β-mercaptoethanol (Sigma).

Experiments were carried out using the reagents at a molar ratio of SnCe6:EDC:S-NHS of 1:1:2.5.

The pH of the reactive SnCe6 mixture was neutralised to 7.0 by adding 0.7 ml 1 M NaOH. 1.5 ml of phage 75 was then added to the amine-reactive solution to allow the amino groups on the phage to react with the carboxyl groups of the SnCe6, and then mixed for 4 to 16 hours. The reaction was quenched with 2.5 μl ethanolamine (Sigma).

The photosensitiser-phage conjugate (PS-phage) was separated from free PS after conjugation by precipitating the PS-phage twice, as described above in Bacteriophage Precipitation. The PS-phage was then dialysed against PBS.

In the examples below, the concentration of phage 75 is 7.3×10⁶ pfu/ml and the concentration of SnCe6/bacteriophage-SnCe6 is 1.5 μg/ml.

Laser

The laser used was a Model 127 Stabilite helium-neon (He/Ne) laser (Spectra Physics, USA) with a power output of 35 mW. The laser emitted radiation in a collimated beam, diameter 1.25 mm, with a wavelength of 633 nm.

### Example 1

A culture of EMRSA-16 in the mid-exponential growth phase was diluted to 1×10⁷ cfu/ml. 20 μl samples of the diluted bacteria were then placed into wells of a 96-well plate (Nunc), together with a magnetic stirrer bar.

100 μl of the phage 75-SnCe6 conjugate prepared above and calcium chloride (CaCl₂) to a final concentration of 10 mM was added to the bacteria. The contents of the wells were left to incubate at room temperature for 5 min, with stirring. Controls were performed with 100 μl 1×PBS added to the bacteria and used as a reference for experimental samples. The experiment was carried out in duplicate.

After incubation, the contents of the well were directly exposed to the laser light for 5 min, with stirring, corresponding to an energy density of 21 J/cm². Aluminium foil was placed in the surrounding wells to allow any escaping laser light to be reflected back into the target well. Controls were performed with no laser irradiation.

After exposure to the laser, 100 μl samples were immediately taken from each well and serially diluted, from 10⁻¹ to 10⁻⁴, in 1 ml TSY in 1.5 ml Eppendorf tubes. Aliquots of 50 μl of each dilution were then placed and spread out on half a CBA plate. The plates were placed in a 37° C. incubator overnight. The following morning the number of survivors was counted, the average between the four sets was taken and multiplied by the appropriate dilution factor, and graphically analysed.

Phage at 7.3×10⁶ pfu/ml

SnCe6/phage at 1.5 μg/ml

It was found that over 99.9% of the EMRSA-16 were killed.

### Example 2

Example 1 was repeated, using EMRSA-1 in place of EMRSA-16. It was found that 99.98% of the bacteria were killed.

### Example 3

Example 1 was repeated, using EMRSA-3 in place of EMRSA-16. It was found that over 99.99% of the bacteria were killed.

### Example 4

Example 1 was repeated, using EMRSA-15 in place of EMRSA-16. It was found that over 99.99% of the bacteria were killed.

### Example 5

Example 1 was repeated, using S. epidermidis in place of EMRSA-16. It was found that over 99.99% of the bacteria were killed.

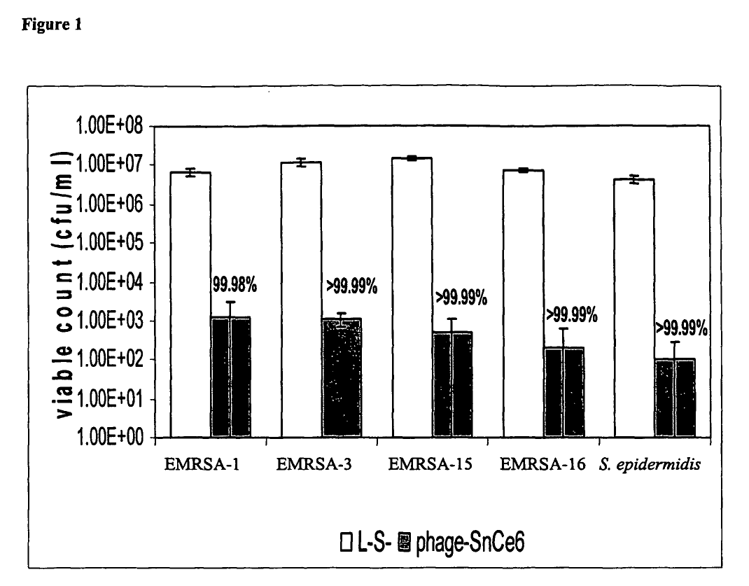

Result for Examples 1 to 5 are presented in FIG. 1.

### Example 6

Example 1 was repeated, using 10 μl each EMRSA-16 and S. epidermidis in place of the 20 μl samples of EMRSA-16. Samples were plated on MBA plates for enumeration.

Phage at 7.3×10⁶ pfu/ml

SnCe6/phage at 1.5 μg/ml

21 J/cm² laser light

It was found that over 99.99% of both bacterial strains were killed in the mixed culture.

### Comparative Example

Example 6 was repeated, firstly in the absence of conjugate, and without exposing to laser light, secondly with SnCe6 photosensitiser and exposure to laser light, and thirdly with phage 75 and without exposure to laser light.

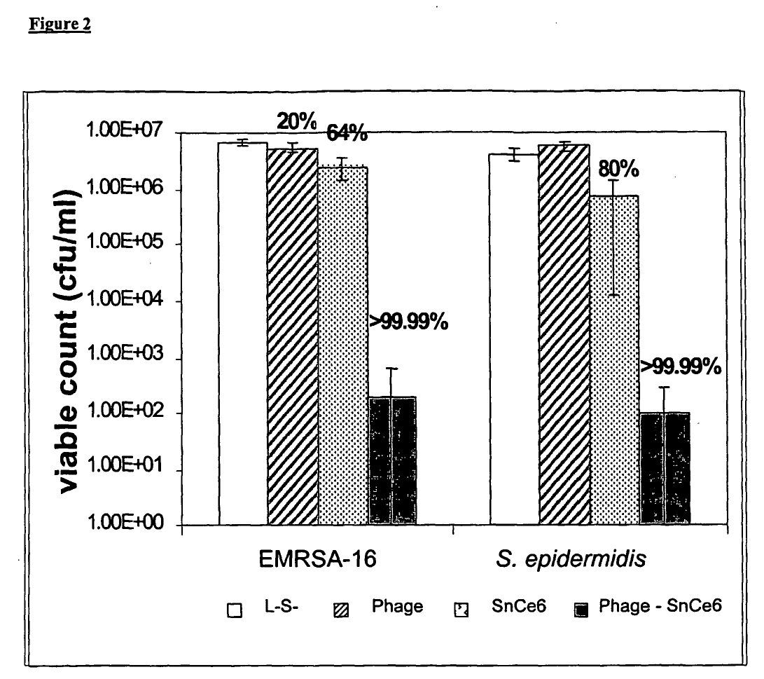

The results for Example 6 and for the Comparative Example are presented in FIG. 2.

The Examples show that the conjugate is highly effective at killing all of the EMRSA strains tested. Since phage 75 is only capable of infecting EMRSA-15 and EMRSA-16, this indicates that the phage is able to successfully bind to strains it is incapable of infecting, thus acting as an effective targetting agent. The attached photosensitisers then effected the killing upon laser irradiation.

Significant kills were also obtained with S. epidermidis, both alone and in a mixture with MRSA, indicating that the phage also bound to non-related staphylococcal strains. The phage 75-SnCe6 conjugate is useful for a variety of staphylococcal infections.

### Example 7

Targeted Photodynamic Therapy Using Φ11-SnCe6 Conjugates Against Staphylococcus aureus and a Laser Light Source

Bacteriophage Φ11 was propagated and precipitated as described above for phage 75, except that S aureus strain 8325-4 was used as the propagating strain. Tin chlorin e6 (SnCe6) was conjugated onto Staphylococcus phage Φ11 using the method described above, achieving bound concentrations of 2.3 and 3.5 μg ml⁻¹ SnCe6 with the phage Φ11 at 4.7×10⁷ pfu.ml⁻¹. These Φ11-SnCe6 conjugates were then incubated with various strains of Staphylococcus aureus and exposed to laser light at 633 nm from a 35 mW HeNe laser (21 J/cm²) for 5 minutes. The final concentration of conjugated SnCe6 was 1.15 μg ml⁻¹.

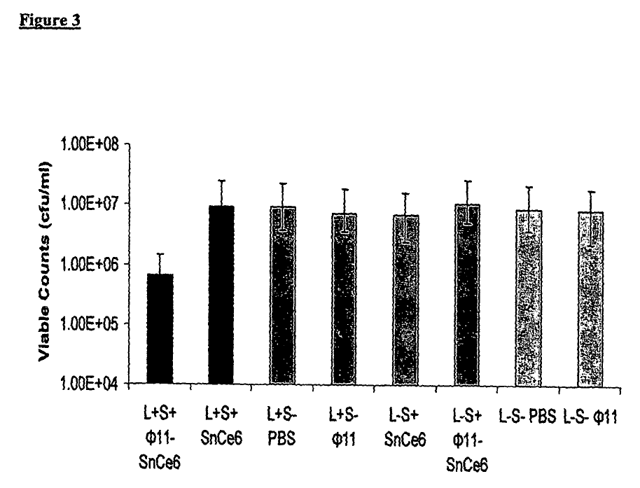

The results show that Φ11-SnCe6 conjugates achieved a 92.33% kill of S. aureus 8325-4 (compared to control counts in phosphate buffered saline) after 5 minutes exposure, whilst SnCe6 at a corresponding concentration (1.15 μg ml⁻¹) did not achieve any kill. The results are presented in FIG. 3.

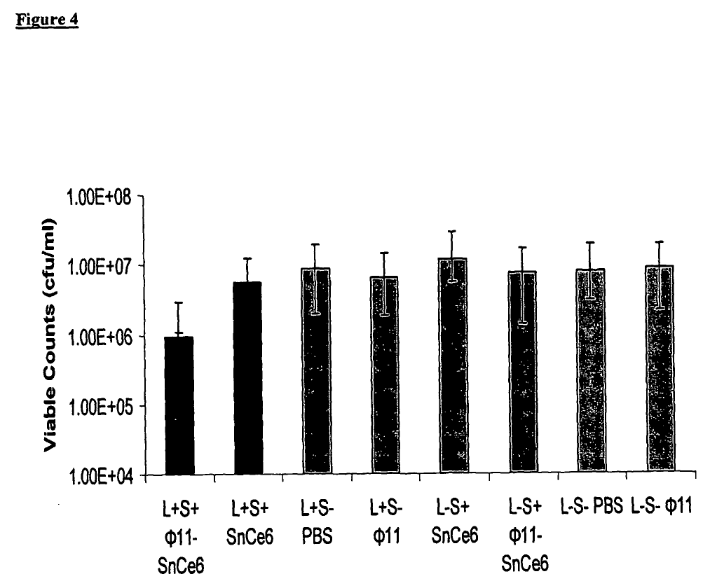

We have also shown that this Φ11-SnCe6 conjugate is effective against a methicillin-resistant strain of the organism (EMRSA-16), achieving 88.11% kill, even though Φ11 only infects this strain under stringent optimal conditions. A range of control experiments such as; light without photosensitiser (L+S−), photosensitiser without light (L−S+), and unconjugated phage at 1×10⁷ pfu ml⁻¹ (L−S−); did not result in significant kills. The results are presented in FIG. 4.

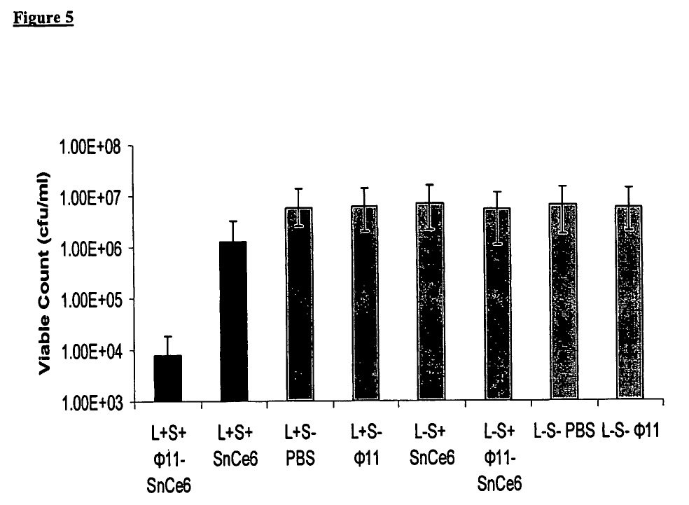

By increasing the light dose to 10 minutes in the presence of calcium (10 mM) we are now achieving 99.88% kills against S. aureus 8325-4 using Φ11-SnCe6 conjugates (1.75 μg ml⁻¹). The results are presented in FIG. 5.

For FIGS. 3 to 5 the photosensitiser (either SnCe6 or Φ11-SnCe6) was added to give a final concentration of 1.15 μg ml⁻¹ (with respect to SnCe6). The light source was a 35 mW Helium/Neon laser and irradiation (when used) was for 5 minutes in the case of FIGS. 3 and 4, and for 10 minutes in the case of FIG. 5.

The effect of varying the light dose on the kills obtained with the SnCe6-phage Φ11 conjugate was investigated. The experiments were carried out as described above except that the bacterial suspensions were exposed to light from the Helium/Neon laser for different periods of time—these were 1, 5, 10, 20 and 30 minutes. In each case, the concentration of the Φ11-SnCe6 conjugate (final concentration equivalent to 3.5 μg ml⁻¹ of SnCe6) was the same.

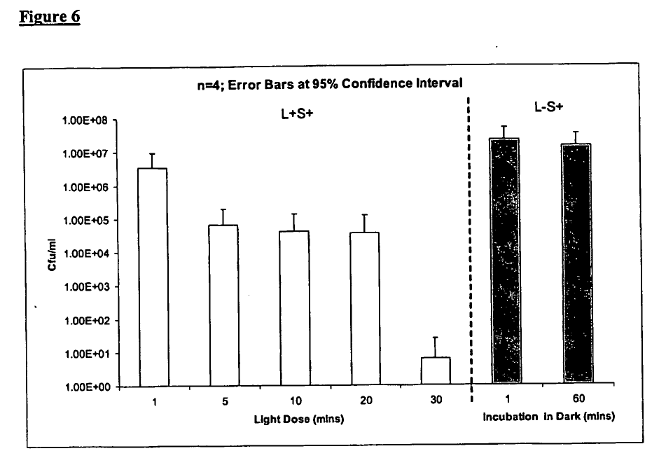

Incubation of the organism with the Φ11-SnCe6 conjugate for up to 60 minutes in the dark had no significant effect on the viable count. However, significant reductions in the viable count were obtained when the suspensions were exposed to laser light in the presence of the Φ11-SnCe6 conjugate—greater kills were obtained with the longer exposure times. Using an exposure time of 30 minutes, a reduction in the viable count of approximately 99.9999% was obtained. Φ11-SnCe6 was used to give a final concentration of 3.5 μg ml⁻¹ (with respect to SnCe6). The light source was a 35 mW Helium/Neon laser and irradiation (when used) was for 1, 5, 10, 20 or 30 minutes. The results are presented in FIG. 6.

In FIGS. 3 to 6
SnCe6=tin chlorin e6
Φ11-SnCe6=tin chlorin e6 conjugated to bacteriophage Φ11
PBS=Phosphate buffered saline
L+S+=bacteria irradiated in the presence of conjugate
L+S−=bacteria irradiated in the absence of conjugate
L−S+=bacteria exposed to conjugate in the absence of light
L−S−=bacteria exposed neither to light nor conjugate

### Example 8

Lethal Photosensitisation of Staphylcoccus aureus Using a phage 75-Tin (IV) Chlorin e6 Conjugate and a White Light Source

Bacterial strains: S. aureus 8325-4
EMRSA-16

Light source: KL200 (Schott). This is a 20-watt halogen cold light source. The light guide attached to it is a flexible optic fibre bundle which is directed onto a 96 well plate at a distance of 5 cm. A square of 4-wells is placed at the centre of the light source.

Approx light intensity=44,000 lux or 470 μW/nm

Phage 75 was conjugated to SnCe6 as described above. Phages were used at a concentration of 1×10⁷ pfu/ml.

Overnight cultures of S. aureus grown in nutrient broth were centrifuged, resuspended in PBS and adjusted to an OD of 0.05 at 600 nm (approximately 4×10⁷ cfu/ml)

50 μl of bacterial culture was aliquoted into a 96-well plate and 50 μl of the one of the following solutions added to the wells:
1) 3.5 μg/ml SnCe6-phage 75 (final concentration 1.75 μg/ml, 1×10⁶ pfu/well) in PBS
2) 1.75 μg/ml SnCe6-phage 75 (final concentration 0.875 μg/ml, 5×10⁵ pfu/well) in PBS
3) 3.5 μg/ml SnCe6 in PBS (final concentration 1.75 μg/ml)
4) 1.75 μg/ml SnCe6 in PBS (final concentration 0.875 μg/ml)
5) PBS
6) Phage 75 at a concentration of 5×10⁵ or 1×10⁶ pfu/well in PBS

Wells were either exposed to white light (4 wells at a time) or wrapped in tin foil and stored in the dark.

After various exposure times an aliquot was taken from each well, serially diluted and spread onto Columbia blood agar. Agar plates were incubated overnight at 37° C. and counted the next day.

Results

TABLE 2

| | | | |
|---|---|---|---|
| Final | | | |
| concentration of | Exposure | L+ S+ SnCe6 | L+S+ phage 75-SnCe6 |
| photosensitiser | time | % kill | % kill |

S. aureus 8325-4

| | | | |
|---|---|---|---|
| 1.75 μg/ml | 10 min | 97.8% | 99.96% |
| 0.875 μg/ml | 10 min | 45.3% | 98.98% |
| 1.75 μg/ml | 20 min | 97.9% | 99.998% |

EMRSA-16

| | | | |
|---|---|---|---|
| 1.75 μg/ml | 10 min | 0% | 99.75% |
| 0.875 μg/ml | 10 min | 0% | 99.69% |
| 1.75 μg/ml | 20 min | 99.78% | 99.997% |

% kill - this is calculated compared to bacteria incubated with PBS and kept in the dark All results are the average of replicate experiments.

Controls included bacteria incubated with SnCe6, phage 75-SnCe6 and phage 75 without exposure to white light. Phage 75 was also exposed to white light. All controls had bacterial counts which were not significantly different to the control suspension which had no photosensitiser added and was not irradiated.

### Example 9

Further tests were carried out on S. aureus strains Mu3, Mu50 and MW2. To suspensions of vancomycin-resistant strains of Staphylococcus aureus (Mu3 and Mu50) or a community-acquired strain of MRSA (MW2), saline, phage 75, SnCe6 or phage 75-SnCe6 was added and samples exposed to light from a 35 mW Helium/Neon laser.

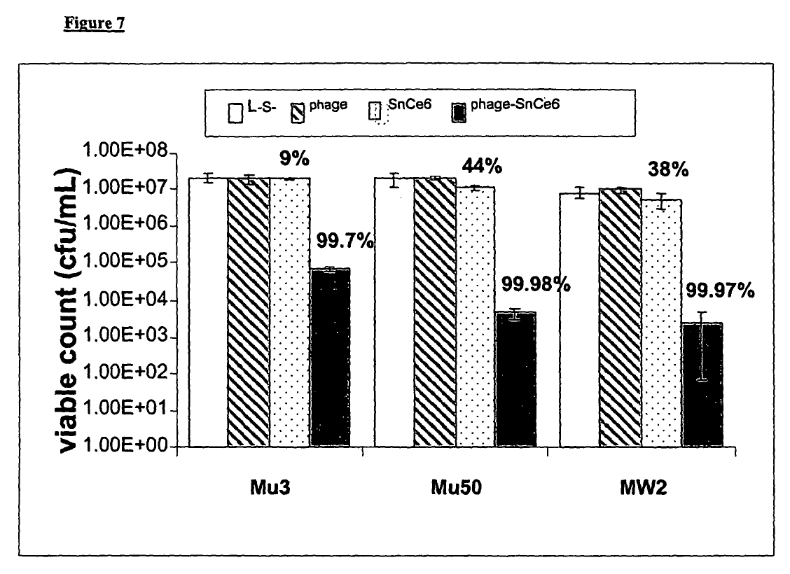

The concentration of SnCe6 used was 1.5 μg/ml, the phage concentration was 5.1×10⁷ plaque-forming units/ml and the light energy dose was 21 J/cm². The numbers above the bars represent the % kill of the organism relative to the sample to which saline only was added. The results are presented in FIG. 7.

### Example 10

Lethal Photosensitization of Streptococcus pyogenes Using Tin chlorin e6 (SnCe6).

streptococcus pyogenes ATCC 12202 was grown in Brain Heart Infusion broth at 37° C. in an atmosphere consisting of 5% CO₂ in air. The cells were harvested by centrifugation and re-suspended in phosphate buffered saline (PBS) and diluted to 1×10⁷ cfu/ml in PBS. 20 μl samples of the diluted bacterial suspension were then placed into wells of a 96-well plate, together with a magnetic stirrer bar. 100 μl of different concentrations (1-50 μg/ml) of the SnCe6 in PBS was added to the bacterial suspensions. Controls were performed with 100 μl PBS added to the bacteria and either irradiated (L+S−) or kept in the dark (L−S−). The experiment was carried out in duplicate.

After incubation, the contents of some of the wells were exposed to light from the 35 mW Helium/Neon laser emitting light with a wavelength of 633nm for 10 min, with stirring, corresponding to an energy density of 42 J/cm². Aluminium foil was placed in the surrounding wells to allow any escaping laser light to be reflected back into the target well. Control wells were not irradiated with laser light.

After exposure to the laser light, 100 μl samples were immediately taken from each well and serially diluted, from 10⁻¹ to 10⁻⁵, in 1 ml TSY in 1.5 ml Eppendorf tubes. Duplicate 50 μl aliquots of each dilution were then spread out on half a CBA plate. The plates were placed in a 37° C. incubator for up to 48 h and the resulting colonies were counted to determine the number of surviving organisms.

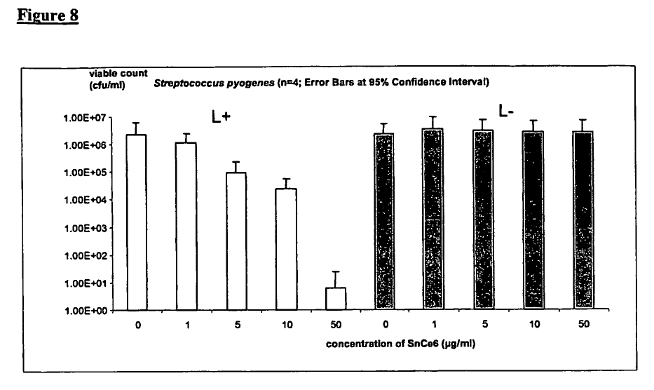

Incubation of the organism in the dark with increasing concentrations of SnCe6 had no significant effect on the viable count. Neither did irradiation of the organism with laser light in the absence of the photosensitiser. However, irradiation of the organism in the presence of SnCe6 resulted in a concentration-dependent decrease in the viable count. A 99.9997% kill of the organism was obtained using a photosensitiser concentration of 50 μg/ml. The results are presented in FIG. 8. In FIG. 8

L+(open bars)=cultures irradiated with laser light in the absence of SnCe6 as well as in the presence of various concentrations of the photosensitiser,

L−(shaded bars)=cultures incubated in the dark in the absence of SnCe6 as well as in the presence of various concentrations of the photosensitiser.

### Example 11

Lethal Photosensitization of Propionibacterium acnes Using Tin Chlorin e6 (SnCe6).

Propionibacterium acnes ATCC 29399 was grown in pre-reduced Brain Heart Infusion broth at 37° C. in an anaerobic atmosphere. The cells were harvested by centrifugation and re-suspended in phosphate buffered saline (PBS) and diluted to 1×10⁸ cfu/ml in PBS. 20 μl samples of the diluted bacterial suspension were then placed into wells of a 96-well plate, together with a magnetic stirrer bar. 100 μl of different concentrations (1-50 μg/ml) of the SnCe6 in PBS was added to the bacterial suspensions. Controls were performed with 100 μl PBS added to the bacteria and either irradiated (L+S−) or kept in the dark (L−S−). The experiment was carried out in duplicate.

After incubation, the contents of some of the wells were exposed to light from the 35 mW Helium/Neon laser emitting light with a wavelength of 633 nm for 10 min, with stirring, corresponding to an energy density of 42 J/cm². Aluminium foil was placed in the surrounding wells to allow any escaping laser light to be reflected back into the target well. Control wells were not irradiated with laser light.

After exposure to the laser light, 100 μl samples were immediately taken from each well and serially diluted, from 10⁻¹ to 10⁻⁵, in 1 ml of pre-reduced TSY in 1.5 ml Eppendorf tubes. Duplicate 50 μl aliquots of each dilution were then spread out on half a CBA plate. The plates were incubated anaerobically at 37° C. and the resulting colonies were counted to determine the number of surviving organisms.

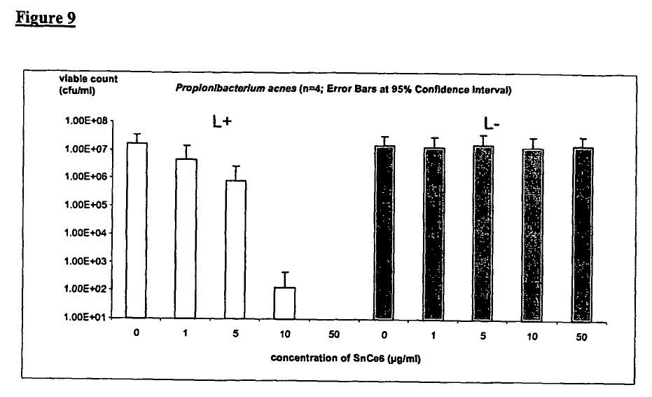

Incubation of the organism in the dark with increasing concentrations of SnCe6 had no significant effect on the viable count. Neither did irradiation of the organism with laser light in the absence of the photosensitiser. However, irradiation of the organism in the presence of SnCe6 resulted in a concentration-dependent decrease in the viable count. A 100% kill of the organism was obtained using a photosensitiser concentration of 50 μg/ml. The results are presented in FIG. 9. In FIG. 9

L+(open bars)=cultures irradiated with laser light in the absence of SnCe6 as well as in the presence of various concentrations of the photosensitiser;

L−(shaded bars)=cultures incubated in the dark in the absence of SnCe6 as well as in the presence of various concentrations of the photosensitiser.

### Example 12

Preparation of Conjugate of TBO and Bacteriophage

1 mg of toluidine blue O (TBO) was dissolved in 800 μl of activation buffer (0.1M MES, 0.5M NaCl pH5.5) together with 0.4 mg EDC and 0.6 mg of S—NHS and 200 μl of phage (5×10⁷ pfu/ml). The reaction was allowed to proceed for 15 to 30 minutes with stirring after which time the EDC was neutralised by adding 1.4 μl of 2-mercaptoethanol. The reaction was allowed to proceed for a further 2 to 4 hours after which time the reaction was quenched by adding hydroxylamine to a final concentration of 10 mM.

The TBO-phage conjugate was separated from free TBO by two rounds of phage precipitation followed by dialysis against PBS.

1. A composition comprising a conjugate of a photosensitiser and a bacteriophage.

2. A composition according to claim 1, wherein the bacteriophage is a staphylococcal bacteriophage.

3. A composition according to claim 1, wherein the photosensitiser is covalently linked to the bacteriophage.

4. A composition according to claim 1, wherein the photosensitiser is chosen from Porphyrins, phthalocyanines, chlorins, bacteriochlorins, phenothiaziniums, phenazines, acridines, texaphyrins, cyanines, anthracyclins, pheophorbides, sapphyrins, fullerene, halogenated xanthenes, perylenequinonoid pigments, gilvocarcins, terthiophenes, benzophenanthridines, psoralens and riboflavin.

5. A composition according to claim 4, wherein the photosensitiser is tin (IV) chlorin e6 (SnCe6).

6. A composition according to claim 1, wherein the bacteriophage is chosen from phage 53, 75, 79, 80, 83, φ11, φ12, φ13, φ147, φMR11, 48, 71, φ812, SK311, φ131, SB-I, U16, C₁, SF370.1, SP24, SFL, A1, ATCC 12202-B1, f304L, φ304S, φ15, φ16, 782, P1c1r100KM, P1, T1, T3, T4, T7 MS2, P1, M13, UNL-1, ACQ, UT1, tba1D3, E79, F8, pf20 B3, F116, G101, B86, T7M, ACq, UT1, BLB, PP7, ATCC 29399-B1 and B40-8.

7. A composition according to claim 6, wherein the bacteriophage is phage 75 or phage Φ11.

8. A composition according to claim 1, wherein the concentration of the photosensitiser is from 0.01 to 200 μg/ml.

9. A composition according to claim 1, wherein the concentration of the bacteriophage is from 1×10⁵ to 1×10¹⁰ pfu/ml.

10. A composition according to claim 1, which further comprises a source of Ca²+ ions, preferably calcium chloride.

11. A composition according to claim 1, in the form of a solution in a pharmaceutically acceptable carrier.

12. A composition according to claim 1, wherein the composition further comprises one or more of a buffer, salt, antioxidant, preservative, gelling agent or remineralisation agent.

13. A method of killing bacteria, comprising
(a) contacting an area to be treated with a composition according to claim 1, such that any bacteria present bind to the photosensitiser-bacteriophage conjugate; and
(b) irradiating the area with light at a wavelength absorbed by the photosensitiser.

14. A method according to claim 13, wherein the bacteria are staphylococcus, particularly MRSA, EMRSA VRSA, hetero-VRSA or CA-MRSA.

15. A method according to claim 13, wherein the light is laser light or white light.

16. A method according to claim 15, wherein the laser light is from a helium neon gas laser.

17. A method according to claim 15, wherein the laser light has a wavelength of from 200 to 1060 nm.

18. A method according to claim 15, wherein the laser has a power of from 1 to 100 mW and a beam diameter of from 1 to 10 mm.

19. A method according to claim 18, wherein the light dose of laser irradiation is from 5 to 333 Jcm⁻².

20. A method according to claim 15, wherein the light dose of white light is from 0.01 to 100 J/cm².

21. A method according to claim 15, wherein the duration of irradiation is form one second to 15 minutes.

22. A method according to claim 13, wherein the composition is present in or on the area to be treated at a concentration of from 0.00001 to 1% w/v.

23. Use of a composition according to claim 1, for treatment of the human or animal body.

24. Use of a composition according to claim 1, in the manufacture of a medicament for treatment of bacterial infection.

25. Use according to claim 24, wherein the bacterial infection is S. aureus, particularly MRSA, EMRSA, VRSA, hetero-VRSA or CAMRSA.

26. Use of a bacteriophage as a targeting agent in photodynamic therapy (PDT).

27. Use according to claim 26, wherein the bacteriophage is a staphylococcal phage.

28. A composition according to claim 1, substantially as described in the Examples.

29. A method according to claim 13, substantially as describe in the Examples.

30. A use according to claim 23, substantially as described in the Examples.
